(19) United States
(12) Patent Application Publication
(10) Pub. No.: US 2017/0105933 A1
(43) Pub. Date: Apr. 20, 2017

(54) Suppression of Undesired 18 FDG Uptake within inflammatory lymph nodes and inflamed tissues on Positron Emission Tomography Scans that are obtained for Neoplastic Staging

(71) Applicant: Michal Kulon, East Setauket, NY (US)

(72) Inventor: Michal Kulon, East Setauket, NY (US)

(21) Appl. No.: 14/918,352

(22) Filed: Oct. 20, 2015

(51) Int. Cl. A61K 9/00

(52) U.S. Cl. CPC A61K 9/0053; A61K 9/0019; A61K 9/0014

(57) ABSTRACT

Positron Emission Tomography (PET, PET-CT, or PET-MRI) scans utilizing ¹⁸FDG (fluorodeoxyglucose) are often performed for the evaluation of the extent of neoplastic disease, specifically to assess for the extent of metastases, in order to guide cancer staging and treatment. ¹⁸FDG is typically avidly taken up by neoplastic cells, but also by inflamed tissues such as lymph nodes. In th context of neoplastic staging, such inflammatory uptake is often undesired, and leads to occasional uncertainty in the assessment of the extent of metastatic disease if inflammatory processes are concurrently present. A regimen consisting of one or more anti-inflammatory medications administered prior to PET scan can potentially suppress such undesired inflammatory FDG activity. Administration of such medication regimen could be routine, before a subset or entirety of all PET scan attempts for certain groups of diseases, or prior to a repeat PET scan, used to resolve difficult cases, leading to 2 PET scans, one with and one without the anti-inflammatory medication.

### FIELD OF THE INVENTION

This invention relates to methods of using an anti-inflammatory medication to suppress undesired uptake of ¹⁸FDG within inflammatory processes on Positron Emission Tomography scans which were performed for the purpose of neoplastic staging.

### BACKGROUND OF THE INVENTION

Positron Emission Tomography scans (PET), or combined with another modality (such as PET-CT or PET-MRI) utilizing ¹⁸FDG (fluorodeoxyglucose) are often performed for the evaluation of the extent of neoplastic disease, specifically to assess for the number and extent of metastases, in order to guide cancer staging and treatment. ¹⁸FDG is typically avidly taken up by neoplastic cells, but also by inflamed tissues such as inflamed lymph nodes. In the context of neoplastic staging, such inflammatory uptake is undesired, and can occasionally lead to uncertainty in the assessment of the extent of metastatic disease if inflammatory processes are concurrently present (such as in the presence of pneumonia, infectious diseases, or immunological diseases). Anti-inflammatory medication can be administered prior to PET scan to suppress such undesired inflammatory FDG activity, or to suppress this relative to the primary neoplasm. Multiple classes of anti-inflammatory medications exist, each class with multiple individual medications. These anti-inflammatory medications often have similar effects, although vary in effective dosages. Such anti-inflammatory medications include aspirin, NSAIDS (such as naproxen, ibuprofen, diclofenac, indomethacin, ketorolac, ketoprofen, etc), COX-2 inhibitors (such as celecoxib, etoricoxib, parecoxib), or steroids (such as Cortisone, Hydrocortisone, Methylprednisolone, Prednisolone, Prednisone, Triamcinolone, Betamethasone, Dexamethasone). Administration of such medication regimen could be routinely done prior to PET scan attempts for certain groups of diseases, or prior to a repeat PET scan, to resolve difficult cases, leading to 2 PET scans, one with and one without the anti-inflammatory medication.

### BRIEF SUMMARY OF THE INVENTION

Priority is claimed based on Provisional application Ser. No. 10/14/2015, Application No. 62/241,411.

The present invention relates to the use of one or more anti-inflammatory medications administered prior to PET, PET-CT or PET-MRI scans utilizing FDG that are performed for assessment of the extent of neoplastic disease, to suppress undesired inflammatory activity.

### DETAILED DESCRIPTION OF THE PREFERRED EMBODIMENTS

Purpose of the invention is to suppress undesired FDG uptake by inflammatory processes on PET scans by means of one or more anti-inflammatory medications, including aspirin, NSAIDS (such as naproxen, ibuprofen, diclofenac, indomethacin, ketorolac, ketoprofen), COX-2 inhibitors (such as celecoxib, etoricoxib, parecoxib), and steroids (such as Cortisone, Hydrocortisone, Methylprednisolone, Prednisolone, Prednisone, Triamcinolone, Betamethasone, Dexamethasone). The methods comprise administering an anti-inflammatory medication via oral, intravenous, or transdermal route at one or more time-points prior to PET scan. This may be performed routinely for nearly all patients, or restricted to a subset of neoplastic diseases, or used only if there is a suspicion of a concomitant inflammatory process.

I claim:

1. A method for diminishing undesired ¹⁸FDG (fluorodeoxyglucose) uptake in inflammatory processes and inflamed lymph nodes on Positron Emission Tomography scan, comprising administration of one or more anti-inflammatory medications prior to scan.

2. Claim 1, wherein the said Positron Emission Tomography scan is one from a set comprising PET, attenuation-corrected PET, PET-CT, or PET-MRI scan.

3. Claim 2, wherein the anti-inflammatory medication is routinely administered prior to scan to patients whose disease is a disease from a subcategory of neoplastic diseases, except for patients with a medical contra-indication.

4. Claims 2, wherein the anti-inflammatory medication is administered prior to a repeat scan, to resolve an ambiguous PET scan result.

5. Claim 2, wherein the anti-inflammatory medication is administered to patients who are suspected of having a plurality of concomitant diseases from a set of disease categories comprising neoplastic, inflammatory, infectious diseases.

6. Claim 2, wherein the anti-inflammatory medication is administered via oral route.

7. Claim 2, wherein the anti-inflammatory medication is administered via intravenous route, comprising a syringe.

8. Claim 2, wherein the anti-inflammatory medication is administered via subcutaneous route, comprising a syringe and needle.

9. Claim 2, wherein the anti-inflammatory medication is administered via transdermal route, comprising a medication patch.

10. Claim 2, wherein the anti-inflammatory medication is from a set of Non-steroidal anti-inflammatory NSAIDs.

11. Claim 2, wherein the anti-inflammatory medication is from a set of COX-2 inhibitors.

12. Claim 2, wherein the anti-inflammatory medication is from a set of steroids.

13. Claim 2, wherein the medication is one or plurality from a set comprising aspirin, diclofenac, indomethacin, etodolac, sulindac, tolmetin, ketorolac, ibuprofen, ketoprofen, dexketoprofen, naproxen, fenoprofen, flurbiprofen, oxaprozin, piroxicam, meloxicam, lornoxicam, tenoxicam, mefenamic acid, meclofenamate, niflumic acid, tolfenamic acid, flufenamic acid, metamizole, phenazone, aminopyrine, propyphenazone, phenylbutazone, nimesulide, nabumetone, celecoxib, etoricoxib, parecoxib, Cortisone, Hydrocortisone, Methylprednisolone, Prednisolone, Prednisone, Triamcinolone, Betamethasone, Dexamethasone.

14. Claim 2, wherein the anti-inflammatory medication is administered several times, over multiple hours prior to scan.

15. Claim 2, wherein the anti-inflammatory medication is administered several times, over multiple days prior to scan.

16. Claim 6, further comprising an element from a set of elements comprising: tablet, caplet, liquid, powder, gel, capsule.

17. Claim 2, wherein the said Positron Emission Tomography scan further comprises a scanning device.
